(19) United States
(12) Patent Application Publication
(10) Pub. No.: US 2015/0374216 A1
(43) Pub. Date: Dec. 31, 2015

(54) CAPSULE ENDOSCOPE FOR PHOTODYNAMIC AND SONODYNAMIC THERAPY USING MAGNETISM

(71) Applicant: CATHOLIC UNIVERSITY INDUSTRY ACADEMIC COOPERATION FOUNDATION, Seocho-gu, Seoul (KR)

(72) Inventor: Hiun Suk CHAE, Seoul (KR)

(73) Assignee: CATHOLIC UNIVERSITY INDUSTRY ACADEMIC COOPERATION FOUNDATION, Seocho-gu, Seoul (KR)

(21) Appl. No.: 14/769,589

(22) PCT Filed: Dec. 5, 2013
(86) PCT No.: PCT/KR2013/011149

§ 371 Date: Aug. 21, 2015

(30) Foreign Application Priority Data
Feb. 21, 2013 (KR) 10-2013-0018750

(51) Int. Cl. A61B 1/04; A61N 7/00; A61B 1/045; A61N 5/06

(52) U.S. Cl. CPC A61B 1/041; A61N 5/0601; A61N 5/062; A61N 7/00; A61B 1/045; A61N 2007/0043

(57) ABSTRACT

The present invention relates to a capsule endoscope for a photodynamic and sonodynamic therapy using magnetism. The capsule endoscope for a photodynamic and sonodynamic therapy using magnetism includes: an endoscope body; a light and ultrasonic wave irradiation part provided on an outer surface of the endoscope body, for irradiating light and ultrasonic waves having different wavelengths; and a photodynamic and sonodynamic therapy part connected to an outer surface of the endoscope body and including materials for a photodynamic and ultrasonic therapy, and the endoscope body has a positive electrode part and a negative electrode part having different polarities and is controlled such that the polarities are changed, and the photodynamic and sonodynamic therapy part is activated by photosensitizers leaked from a therapy part connected to the endoscope by light and ultrasonic waves irradiated by the light and ultrasonic wave irradiation part.

### TECHNICAL FIELD

The present invention relates to a capsule endoscope, and more particularly to a capsule endoscope for a photodynamic and sonodynamic therapy using magnetism which is inserted into the interior of the body to irradiate light and ultrasonic waves of a specific wavelength for treatment of infection of local pathogenic bacteria in order to be used for diagnosis and treatment of a gastro-intestinal tract or a limited body portion.

### BACKGROUND ART

For the past, antibiotics have been mainly used for treatment of many pathogenic bacteria including germs.

However, the death rate due to infection by pathogenic bacteria such as germs (so called super bacteria) representing resistance to several antibiotics used recently, and composition of gut microbes is related to the cause of chronic IBDs and generation of malignant diseases of gastro-intestinal tracts.

For example, problems of resistant germs that are resistant to antibiotics such as metronidazole, vancomycin, and Clathramycin in the case of Helicobacter pylori or Clostridium difficile infection in infection of a digestive system have been raised. Another problem of the treatment of antibiotics in infection of germs is the compliance of a patient, and patients may not take antibiotics due to allergies and gastrointestinal trouble and bitter taste against the antibiotics. Accordingly, a therapy that can replace antibiotics for treatment of pathogenic bacteria is required and acute or chronic diseases due to pathogenic bacteria of a digestive system can be treated through development of the therapy, and ultimately, the therapies are helpful to chronic gastroenteritis and malignant tumors of the gastro-intestinal tracts.

The photodynamic therapy (PDT) and the sonodynamic therapy (SDT) using light and ultrasonic waves are therapies used in the case of malignant tumors or local germ infections and is a method for curing pathogenic bacteria using photosensitizers (PSs) having a specific wavelength and shows a sterilization effect and an anti-tumor effect as reactive oxygen species (ROS) is generated by operations of light, ultrasonic waves, and the PS to cause damage to cell walls, cell membranes, and nucleic acids.

The sensitizers are divided into two types, and are classified into portoporphyrin based PSs and nonprotoporphyrin PSs. The therapies are mainly used for treatment of infection that is spatially limited, and are mainly used for the dermatological or dental field. Because pathogenic bacteria living in human body have not been exposed to light for a long time, many repairing genes to light injury are retrogressed evolutionally. And genes resistant to the photo-therapy have not be discovered until now.

Accordingly, the photodynamic and sonodynamic therapy for the pathogenic bacteria of the body will be very effective, and generation of antibiotics resistant germs can be economically reduced by reducing use of antibiotics. Furthermore, because the PDTs and the SDTs are used first before introduction of antibiotics, at least bacterial loading can be reduced and thus death rate due to infection of pathogenic bacteria can be reduced in addition to the economic effect.

According to the related art, the capsule endoscopes inserted into the body have been used only for diagnosis. Korean Patent Application Publication No. 2004-0108277 and Korean Patent Application Publication No. 2010-0069192 disclose capsule endoscopes.

Accordingly, the present invention suggests a capsule endoscope that can be utilized for the above-mentioned photodynamic and sonodynamic therapy without being limited to a diagnosis.

### DISCLOSURE OF THE INVENTION

### Technical Problem

Accordingly, the present invention has been made in an effort to solve the above-mentioned problems, and for example, is inserted into a stomach for treatment of infection of a stomach to irradiate light and ultrasonic waves of a specific wavelength and activate a PS. In addition, the present invention provides a capsule endoscope for a photodynamic and sonodynamic therapy using magnetism.

The technical objects of the present invention are not limited to the above-ones, and the other unmentioned technical objects will be clearly understood by those skilled in the art from the following description.

### Technical Solution

In accordance with an aspect of the present invention, there is provide a capsule endoscope for a photodynamic and sonodynamic therapy using magnetism, the capsule endoscope including: an endoscope body; a light and ultrasonic wave irradiation part provided on an outer surface of the endoscope body, for irradiating light and ultrasonic waves having different wavelengths; and a photodynamic and sonodynamic therapy part connected to an outer surface of the endoscope body and including materials for a photodynamic and ultrasonic therapy, wherein each half of the endoscope body has a positive electrode part and a negative electrode part having different polarities and is controlled such that the polarities are changed periodically, and the photodynamic and sonodynamic therapy part is activated by photosensitizers leaked from a therapy part connected to the endoscope by light and ultrasonic waves irradiated by the light and ultrasonic wave irradiation part.

The positive electrode part and the negative electrode part are controlled such that the polarities thereof are changed at a predetermined time interval.

The endoscope body is formed of a metal material, and the endoscope body is controlled to be switched on and off such that the endoscope body has or does not have magnetism outside the body.

The photodynamic and sonodynamic therapy part is connected to the endoscope body by a connection fiber decomposed in a pH condition or other conditions (germ ferments and the like) according to the pathophysiology of diseases.

The light and ultrasonic wave irradiation part has a boss shape protruding from an outer surface of the endoscope body.

The endoscope body has a spherical shape.

The light and ultrasonic wave therapy part has a form of a microcapsule, and includes light and ultrasonic wave sensitizers, antibiotics, nanomaterials, and lactobacilli.

Cameras are provided at a front end and a rear end of the endoscope body.

The light and ultrasonic wave irradiation part and the cameras are controlled to be switched on and off outside the body.

In accordance with another aspect of the present invention, there is provided a photodynamic and sonodynamic therapy apparatus comprising: a capsule endoscope; and a magnet rearranged to a desired location by pulling the endoscope body from the outside of a body when the endoscope body is controlled not to have magnetism.

### Advantageous Effects

The present invention provides a capsule endoscope for a photodynamic and sonodynamic therapy that shows an antibacterial effect by reactive oxygen species (ROS) by irradiating light and ultrasonic waves of a specific wavelength and activating photosensitizers separated from a fiber connected to the endoscope with the light and ultrasonic waves irradiated from the endoscope body.

Accordingly, because a diagnostic operation that is an essential function of the capsule endoscope and a treatment can be performed at the same time while the capsule endoscope passes through the body, a separate therapy is not necessary and a patient can conveniently receive a treatment.

In addition, when the capsule endoscope having magnetism is used, the capsule endoscope moved to the vicinity of a duodenum can be moved to a desired location again using an external magnet and can perform treatment of PDT repeatedly.

### BRIEF DESCRIPTION OF THE DRAWINGS

FIG. 1 is a diagram illustrating a capsule endoscope for a photodynamic and sonodynamic therapy using magnetism according to an embodiment of the present invention.

FIG. 2 is a diagram schematically illustrating that a capsule endoscope is rearranged through a magnet outside the human body according to an embodiment of the present invention.

### BEST MODE FOR THE INVENTION

Hereinafter, a capsule endoscope for a photodynamic and sonodynamic therapy using magnetism according to an embodiment of the present invention will be described in detail with reference to the accompanying drawings.

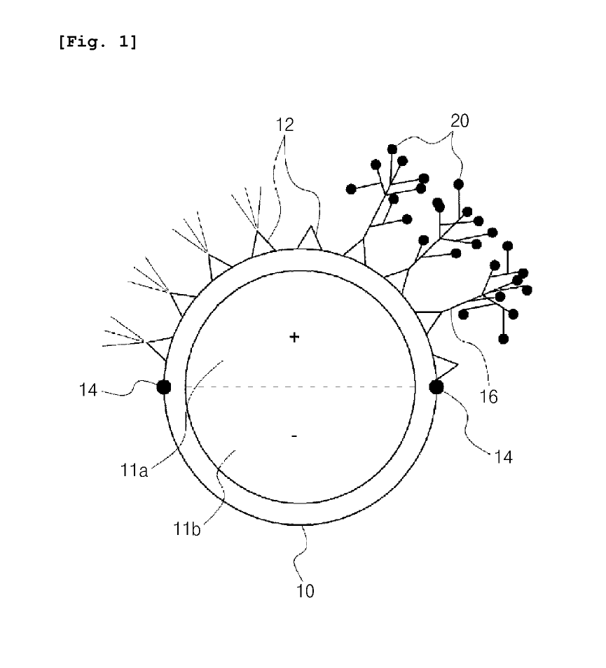

FIG. 1 is a diagram illustrating a capsule endoscope for a photodynamic and sonodynamic therapy using magnetism according to an embodiment of the present invention.

Referring to the drawing, the capsule endoscope for a photodynamic and sonodynamic therapy according to the present invention includes: an endoscope body 10; a light and ultrasonic wave irradiation part 12 provided on an outer surface of the endoscope body 10, for irradiating light and ultrasonic waves having different wavelengths; and a photodynamic and sonodynamic therapy part 20 connected to an outer surface of the endoscope body 10 and including materials for a photodynamic and ultrasonic therapy. The capsule endoscope may be used in a limited space of the body, for example, organs such as a gastro-intestinal tract, a urinary bladder, and an articulation.

As illustrated in FIG. 1, the endoscope body 10 corresponds to a spherical capsule. Of course, the endoscope body 10 may have various shapes such as a cylindrical shape and a rectangular shape as well as the shape illustrated in

FIG. 1. That is, the endoscope body 10 may have any capsule shape that may be inserted into the body.

Meanwhile, in the embodiment of the present invention, the endoscope body 10 is divided into a positive electrode party 11a and a negative electrode part 11b such that the positive electrode part 11a and the negative electrode part 11b occupy halves of the endoscope body 10, respectively, and the polarities of the positive electrode part 11a and the negative electrode part 11b are reciprocally interchangeable at a predetermined time interval so that several capsule endoscopes may effectively move around freely the mucous membrane of a stomach to irradiate light and ultrasonic waves by a pulling or pushing force among multiple capsules. Because several wrinkles (folds) are formed on a surface of the stomach so that the surface of the stomach is uneven and valleys are formed between the winkles so that a desired PS or light cannot reach efficiently the surface of the stomach, the valleys between the wrinkles are widened by a pushing or pulling force between the capsule endoscopes to reach the PS or light sufficiently to the mucous membrane. Of course, the capsule endoscope may be introduced into other organs (for example, a urinary bladder or an articulation) as well as into a stomach. Accordingly, the endoscope body 10 is formed in this way so that the polarities of a plurality of capsule endoscopes introduced into the body can be automatically changed to efficiently go around in the body by a pulling or pushing force.

In particular, among organs of the body, a stomach has a mucous membrane and many folds, so that the capsule endoscopes cannot be go around freely. Then, a plurality of light and ultrasonic wave irradiation parts 12 are provided on an outer surface of the endoscope body 10, and the light and ultrasonic wave irradiation parts 12 have boss shapes protruding from an outer surface of the endoscope body 10. A plurality of light and ultrasonic wave irradiation parts 12 are provided to irradiate light and ultrasonic waves having several different wavelengths according to the PS. The light and ultrasonic waves irradiated from the light and ultrasonic wave irradiation pats 12 activate light and ultrasonic photosensitizers to show a photodynamic and sonodynamic effect for curing pathogenic bacteria in the body. Here, the light and ultrasonic wave irradiation parts 12 have different wavelengths to activate various light and ultrasonic photosensitizers and cure pathogenic bacteria of various kinds.

The light and ultrasonic wave irradiation parts 12 may be controlled such that irradiation of light and ultrasonic waves are switched on and off through a controller (not illustrated) separately provided outside the body. For example, a signal is transmitted to the light and ultrasonic wave irradiation parts 12 so that light and ultrasonic waves having wavelengths suitable for activating PS to kill corresponding bacteria in an area that requires treatment while the interior of the body is observed through a camera that will be described below. In this way, the capsule endoscope is inserted into the body and the interior of the body is observed form the outside in real time so that various bacteria can be selectively treated.

Meanwhile, the cameras 14 are provided at a front end and a rear end of the endoscope body 10. The camera 14 functions to irradiate light while photographing a surface of the mucous membrane of a gastro-intestinal tract as used in a general capsule endoscope. Although FIG. 1 illustrates that the camera 14 is provided at a front end and a rear end of the endoscope body 10, the present invention is not limited thereto but may be provided at various parts such as a side part of the endoscope body 10. The camera 14 may be controlled to be switched on and off by the controller provided outside the body like the light and ultrasonic wave irradiation part 12.

A plurality of connection fibers 16 are provided on an outer surface of the endoscope body 10. The connection fibers 16 function as media that connect the photodynamic and sonodynamic therapy part 20 to the endoscope body 10, and may be resiliently provided on an outer surface of the endoscope body 10. In the embodiment of the present invention, it is preferable that the connection fibers 16 are decomposed in a specific condition (a specific pH, a germ ferment-urease, and the like). This is because the connection fibers 16 are decomposed from the photodynamic and sonodynamic therapy parts 20 to be completely separated from the endoscope body 10 and transferred to a desired portion. For example, the connection fibers 16 may be formed to be decomposed at a specific pH of the stomach. The connection fibers 16 may be decomposed by several materials secreted form germs, and organs and tissues of human body according to the pathophysiology of diseases.

Meanwhile, the photodynamic and sonodynamic therapy part 20 has the form of a microcapsule, and may include nanomaterials such as photosensitizers, antibiotics, and Chitosan. As described above, in the photodynamic and sonodynamic therapy part 20, the connection fibers 16 are decomposed to be separated from the endoscope body 10 and transferred to a desired portion, and an antibacterial function is shown by the reactive oxygen species (ROS) generated when the photosensitizers are activated by the light and ultrasonic waves irradiated from the light and ultrasonic wave irradiation part 12. Because the microcapsules are also decomposed according to specific conditions (for example, gastroenteritis or Hlicobacter pylori infections) of organs, the contained materials are leaked out.

Furthermore, according to special cases, proton pump inhibitors may be used for preprocessing to convert the pH in the stomach into alkali in order to show a strongest effect.

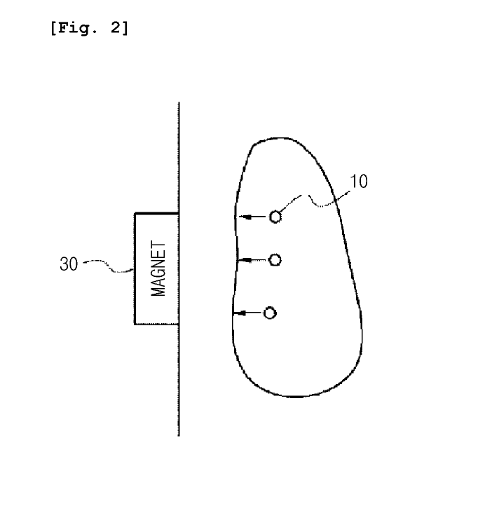

Next, FIG. 2 is a diagram schematically illustrating that a capsule endoscope is rearranged through a magnet outside the human body according to an embodiment of the present invention.

Referring to the drawing, the endoscope body 10 of the endoscope body according to the present invention may be formed of a metallic material. The endoscope body 10 may be controlled to be switched on or off so as not to have magnetism outside the body. As a result, if the endoscope body 10 has magnetism, the polarities of the positive electrode part 11a and the negative electrode part 11b of the endoscope body 10 are changed at a predetermined time interval to show a force for pushing or pulling another capsule endoscope. Meanwhile, if the endoscope body 10 is controlled not to have magnetism, the endoscope body 10 of a metal material may be rearranged at a desired location by a magnet 30 having a strong magnetic field, by which the endoscope body 10 can be freely adjusted from the outside of the body.

For example, when the therapy is intended to be repeatedly performed after one photodynamic and sonodynamic therapy is performed in an on state in the case in which the endoscope body is introduced into the stomach, the endoscope body 10 is converted into an off state and the magnet 30 is located at a border of an esophagus and a stomach to prevent the endoscope body 10 from moving into a duodenum so that the plurality of endoscope bodies 10 that moved to the vicinity of the duodenum may gather near the stomach and the esophagus to resume a desired photodynamic therapy.

A plurality of single capsule endoscopes for a photodynamic and sonodynamic therapy illustrated in FIG. 1 may be introduced at the same time as illustrated in FIG. 2. A plurality of capsule endoscopes are introduced to compensate for the weak antibacterial effect of a single capsule endoscope, and a suitable number of capsule endoscopes may be inserted into the body according to the state of a patient.

It is preferable that the photodynamic and sonodynamic therapy is designed to be applied mainly to a gastrointestinal tract.

Accordingly, in the embodiment of the present invention, in particular, the photodynamic and sonodynamic capsule endoscope is designed to be moved by a magnet outside the body in a limited space of the body and such that a photodynamic and sonodynamic therapy may be repeated performed at a desired location. Of course, the photodynamic and sonodynamic capsule endoscope may be designed to perform a photodynamic and sonodynamic therapy in another organ through control according to the conditions of the organs.

The scope of the present invention is not limited to the above-described embodiment and is defined by the claims, and it will be understood that the present invention may be variously modified and changed by those skilled in the art without departing from the scope of the present invention.

1. A capsule endoscope for a photodynamic and sonodynamic therapy using magnetism, the capsule endoscope comprising:
an endoscope body;
a light and ultrasonic wave irradiation part provided on an outer surface of the endoscope body, for irradiating light and ultrasonic waves having different wavelengths; and
a photodynamic and sonodynamic therapy part connected to an outer surface of the endoscope body and including materials for a photodynamic and ultrasonic therapy, wherein the endoscope body has a positive electrode part and a negative
electrode part having different polarities and is controlled such that the polarities are changed, and the photodynamic and sonodynamic therapy part is activated by light and ultrasonic waves irradiated by the light and ultrasonic wave irradiation part.

2. The capsule endoscope of claim 1, wherein the positive electrode part and the negative electrode part are controlled such that the polarities thereof are changed at a predetermined time interval.

3. The capsule endoscope of claim 1, wherein the endoscope body is formed of a metal material, and the endoscope body is controlled to be switched on and off such that the endoscope body has or does not have magnetism outside the body.

4. The capsule endoscope of claim 1, wherein the photodynamic and sonodynamic therapy part is connected to the endoscope body by a connection fiber decomposed in a pH condition or other conditions according to the pathophysiology of diseases.

5. The capsule endoscope of claim 1, wherein the light and ultrasonic wave irradiation part has a boss shape protruding from an outer surface of the endoscope body.

6. The capsule endoscope of claim 1, wherein the endoscope body has a spherical shape.

7. The capsule endoscope of claim 1, wherein the light and ultrasonic wave therapy part has a form of a microcapsule, and includes light and ultrasonic wave sensitizers, antibiotics, nanomaterials, and lactobacilli.

8. The capsule endoscope of claim 1, wherein cameras are provided at a front end and a rear end of the endoscope body.

9. The capsule endoscope of claim 1, wherein the light and ultrasonic wave irradiation part and the cameras are controlled to be switched on and off outside the body.

10. A photodynamic and sonodynamic therapy apparatus comprising:
a capsule endoscope of claim 1; and
a magnet rearranged to a desired location by pulling the endoscope body from the outside of a body when the endoscope body is controlled not to have magnetism.

11. A photodynamic and sonodynamic therapy apparatus comprising:
a capsule endoscope of any one of claim 2; and
a magnet rearranged to a desired location by pulling the endoscope body from the outside of a body when the endoscope body is controlled not to have magnetism.

12. A photodynamic and sonodynamic therapy apparatus comprising:
a capsule endoscope of any one of claim 3; and
a magnet rearranged to a desired location by pulling the endoscope body from the outside of a body when the endoscope body is controlled not to have magnetism.

13. A photodynamic and sonodynamic therapy apparatus comprising:
a capsule endoscope of any one of claim 4; and
a magnet rearranged to a desired location by pulling the endoscope body from the outside of a body when the endoscope body is controlled not to have magnetism.

14. A photodynamic and sonodynamic therapy apparatus comprising:
a capsule endoscope of any one of claim 5; and
a magnet rearranged to a desired location by pulling the endoscope body from the outside of a body when the endoscope body is controlled not to have magnetism.

15. A photodynamic and sonodynamic therapy apparatus comprising:
a capsule endoscope of any one of claim 6; and
a magnet rearranged to a desired location by pulling the endoscope body from the outside of a body when the endoscope body is controlled not to have magnetism.

16. A photodynamic and sonodynamic therapy apparatus comprising:
a capsule endoscope of any one of claim 7; and
a magnet rearranged to a desired location by pulling the endoscope body from the outside of a body when the endoscope body is controlled not to have magnetism.

17. A photodynamic and sonodynamic therapy apparatus comprising:
a capsule endoscope of any one of claim 8; and
a magnet rearranged to a desired location by pulling the endoscope body from the outside of a body when the endoscope body is controlled not to have magnetism.

18. A photodynamic and sonodynamic therapy apparatus comprising:
a capsule endoscope of any one of claim 9; and
a magnet rearranged to a desired location by pulling the endoscope body from the outside of a body when the endoscope body is controlled not to have magnetism.
